(19) United States
(12) Patent Application Publication
(10) Pub. No.: US 2016/0186217 A1
(43) Pub. Date: Jun. 30, 2016

(54) METHOD FOR BIOCATALYTIC SYNTHESIS OF SUBSTITUTED OR UNSUBSTITUTED PHENYLACETIC ACIDS AND KETONES HAVING ENZYMES OF MICROBIAL STYRENE DEGRADATION

(71) Applicant: TECHNISCHE UNIVERSITÄT BERGAKADEMIE FREIBERG, Freiberg (DE)

(72) Inventors: Michel OELSCHLAGEL, Freiberg (DE); Juliane ZIMMERLING, Freiberg (DE); Dirk TISCHLER, Freiberg (DE); Michael SCHLOMANN, Freiberg (DE)

(73) Assignee: TECHNISCHE UNIVERSITAT BERGAKADEMIE FREIBERG, Freiberg (DE)

(21) Appl. No.: 14/897,149

(22) PCT Filed: Jun. 13, 2014
(86) PCT No.: PCT/EP2014/062327

§ 371 Date: Mar. 1, 2016

(30) Foreign Application Priority Data
Jun. 13, 2013 (DE) 10 2013 211 075.8

(51) Int. Cl. C12P 7/40; C12N 9/90; C12P 7/42; C12N 9/02

(52) U.S. Cl. CPC C12P 7/40; C12N 9/0008; C12N 9/0071; C12N 9/90; C12P 7/42; C12Y 102/01039; C12Y 503/99007; C12Y 114/14011

(57) ABSTRACT

The present invention relates to a method for the biocatalytic synthesis of substituted and unsubstituted phenylacetic acids and ketones from styrenes and bicyclic aromatic hydrocarbons using enzymes of microbial styrene degradation in a whole-cell sensor, as well as a kit for the biocatalytic synthesis of substituted and unsubstituted phenylacetic acids and ketones containing a whole-cell catalyst and the use of the method, wherein the method comprises the following steps:
a) providing at least one type of whole-cell catalyst, containing genes which code for the enzymes of styrene degradation and are under the functional control of a regulatable promoter, in an aqueous component,
b) activating the whole-cell catalyst with an inducer and/or an activator, leading to expression of the gene,
c) bringing the activated whole-cell catalyst into contact with a substrate,
d) isolating the reaction products produced, which are advantageously not further metabolized by the whole-cell cat and advantageously accumulate in the aqueous component.

The present invention relates to a method for the biocatalytic synthesis of substituted and unsubstituted phenylacetic acids and ketones from styrenes and bicyclic aromatic hydrocarbons using enzymes of microbial styrene degradation in a whole-cell sensor, as well as to a kit for the biocatalytic synthesis of substituted and unsubstituted phenylacetic acids and ketones containing a whole-cell catalyst, and to the use of the method. The present invention further relates to novel bacterial strains for the biocatalytic synthesis of substituted and unsubstituted phenylacetic acids and ketones from styrenes and bicyclic aromatic hydrocarbons.

Phenylacetic acids and structurally related compounds belong to an industrially important class of compounds. In addition to using compounds of this type as aromas and flavourings (Fahlbusch et al. [Wiley-VCH 2012, 130]), because of their primarily anti-inflammatory, antimycotic and antimicrobial action, they are of great significance in the pharmaceutical and cosmetics industries (Milne et al. [J. Org. Chem. 2011, 76, 9519-9524], Zhu et al. [Food Chem. 2011, 124, 298-302]). α-methyl phenylacetic acids constitute, inter alia, important precursors in the synthesis of hepatitis C-polymerase inhibitors (Wagner et al. [J. Med. Chem. 2009, 52, 1659-1669]) as well as of histamine-2 receptor antagonists (Ghorai et al. [J. Med. Chem. 2008, 51, 7193-7204]). Thus, the anticholinergic compound hyoscyamine can be synthesized from 4-chloro-α-methylphenylacetic acid (Gualtieri et al. [J. Med. Chem. 1994, 37, 1704-1711]). Various methyl-, methoxy-, chloro-, fluoro- as well as bromo-substituted phenylacetic acids are also of application as important precursors for synthesis in pharmaceuticals, for example for the construction of antimycotic dihydrofurans (Pour et al. [Bioorg. Med Chem. 2003, 11, 2843-5866]). 4-fluorophenylacetic acids are used, inter alia, in the production of drugs for disorders of the digestive tract, the nervous system and bladder function (U.S. Pat. No. 7,683,068 B2), as well as for the synthesis of preparations for inhibiting the replication of picornavirus (Hamdouchi et al. [J. Med. Chem. 2003, 46, 4333-4341]).

4-methylphenylacetic acid also constitutes, inter alia, an important precursor for the production of cancer-combatting substances (Luo et al. [Bioorg. Med. Chem. 2011, 19, 6069-6076], Wei et al. [J. Med. Chem. 2007, 50, 3674-3680]). Moreover, phenylacetic acid and its derivatives are essential components in the synthesis of analgesics such as Ibuprofen and Diclofenac as well as as precursors for the synthesis of penicillins. Thus, the naturally occurring penicillin X can be synthesized from p-hydroxyphenylacetic acid (Corse et al. [J. Am. Chem. Soc. 1948, 70, 2837-3843]), whereas the unsubstituted phenylacetic acid is used as a precursor for penicillin G (Douma et al. [Biotechnol. Prog. 2012, 28, 337-348]).

Because of the manifold application possibilities for phenylacetic acid and its derivatives, various chemical syntheses for the production of these compounds have been developed.

U.S. Pat. No. 4,237,314 A discloses a method for the synthesis of phenylacetic acid by the reaction of acetic acid and benzene in the presence of tellurium halide catalysts, wherein temperatures between 100° C. and 200° C. as well as high pressures of up to 15 bar are necessary. In addition to the high economic and ecological disadvantages because of the substantial energy requirements, this variation gives rise to highly toxic and corrosive hydrobromic acid as a by-product.

U.S. Pat. No. 4,220,592 A discloses a two-step method wherein the corresponding phenylacetic acid is produced by hydrolysis of substituted acetonitriles. In the context of this strategy for synthesis, yields of 50% to 95% can be obtained. The high consumption of mineral acids of 60% to 70% by weight and the required reaction temperatures of up to 250° C., however, highlight the considerable ecological and economic disadvantages of this synthesis.

Taqui Khan et al. (J. Mol. Catal 1988, 44, 179-181) and Qui et al. (J. Nat. Gas Chem. 2005, 14, 40-46) disclose a method for the synthesis of phenylacetic acids based on the carbonylation of benzyl chloride using ruthenium(III)-EDTA complexes or 2-chlorobistriphenyl phosphine. Because of the occurrence of by-products and the high requirement for concentrated acid, for ecological reasons, the method cannot be given serious consideration. Similarly, nickel tetracarbonyl or nitrosyltricarbonyl ferrate catalysts can transform benzyl chloride into phenylacetic acid, with yields of approximately 90%. However, this reaction also requires pressures of 10 bar and a temperature of 80° C. (Bertleff [Wiley-VCH 2005, 13 f.]).

Alternatively, phenylacetic acid may also be obtained by the chemical reaction of benzyl alcohol at 175° C. and 71 bar in the presence of rhodium catalysts (Bertleff [Wiley-VCH 2005, 13 f.]).

Chen et al. (J. Org. Chem. 1999, 64, 9704-9710) disclose a multi-stage method for the production of phenylacetic acid derivatives by the chemical reaction of styrenes by means of hydroboration at −66° C. and subsequent homologation at −100° C.; here again, attention should be drawn to the substantial energy consumption during the course of the reaction.

Milne et al. (J. Org. Chem. 2011, 76, 9519-9524) disclose a method for the synthesis of substituted and unsubstituted phenylacetic acids by the iodide-catalysed reduction of the corresponding mendelic acids, wherein yields of 41% to 100% are obtained depending on the reducing agent used. However, the method is time consuming and energy-consuming, since the products have to be isolated after a three-day long reaction at temperatures of 95° C. using a multi-stage preparation method.

Known chemical methods for the synthesis of phenylacetic acid and its derivatives exhibit many major disadvantages, since on the one hand the use of expensive educts, high reaction temperatures and pressures as well as in some cases lengthy and multi-stage methods are disadvantageous from an economics standpoint, and on the other hand the use of large volumes of concentrated acids and bases are onerous from an ecological standpoint. At the same time, in many cases only low yields are obtained, and in some cases the formation of toxic by-products have to be taken into consideration.

Furthermore, in known chemical methods, racemic mixtures are often obtained which have to be separated into their enantiomers, for example for pharmaceutical applications. Thus, selective syntheses are desirable, wherein one enantiomer is formed in great excess.

Chavda et al. (Chirality 2007, 19, 366-373) disclose an elaborate chemical method for the enantioselective synthesis of 2-phenylpropionic acid from tetrahydrofuran and benzophenone.

As an alternative to the manifold chemical methods for the synthesis of phenylacetic acids, there are also some biotechnological methods.

Gilligan et al. (Appl. Microbiol. Biotechnol. 1993, 39, 720-725) disclose the production of (S)-2-phenylpropionic acid with an enantiomeric excess (ee) of 99% by enzymatic transformation of (R,S)-2-phenylpropionitrile with the enzymes nitrile hydratase and amidase from Rhodococcus equi TG328.

An amidase from Agrobacterium tumefaciens d3 transforms racemic 2-phenylpropionamide 95% stereoselectively into (S)-2-phenylpropionic acid, wherein only half of the educt employed is transformed (Trott et al. [Microbiology 2001, 147, 1815-1824]).

Sosedov et al. (Appl. Environ. Microbiol. 2010, 76, 3668-3674) disclose the direct hydrolysis of arylacetonitrile into carbonic acid and ammonia with the aid of an arylacetonitrilase obtained recombinantly from Pseudomonas fluorescens EBC191. However, the arylacetonitriles required are expensive starting materials.

In contrast, styrenes are among the most important industrial educts for the production of industrial quantities of products (including the production of various plastics), which is why they constitute an inexpensive and available alternative to the educts mentioned above. In the USA alone, over 3 million tonnes of styrene were produced in 1990; worldwide production in 1996 was approximately 14.7 million tonnes.

Thus, the aim of the present invention is to provide a biotechnological method for the transformation of substrates into substituted or unsubstituted phenylacetic acids and/or ketones and/or their cyclic derivatives that is ecologically harmless and economically advantageous.

In accordance with the invention, the aim is accomplished by means of a biocatalytic method for the synthesis of a substituted or unsubstituted phenylacetic acid and/or a substituted or unsubstituted ketone and/or a bicyclic derivative in accordance with general formula (I) and/or formula (II)

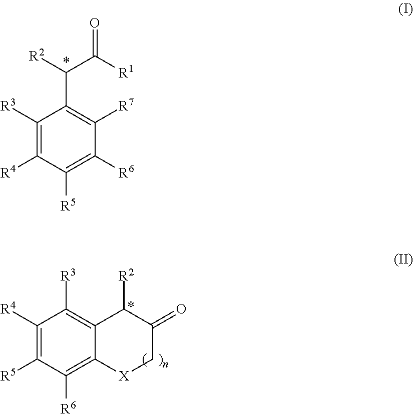

by means of the biocatalytic transformation of a substrate with general formula (III) and/or formula (IV)

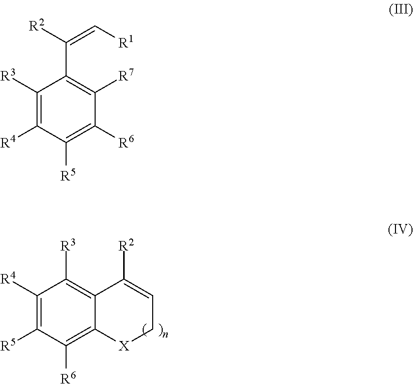

wherein:
the substituent R¹ is H, OH or a linear or branched C₁ to C₃ alkyl residue,
the substituent R² is H or a linear or branched C₁ to C₃ alkyl residue, wherein * is a chiral centre,
the substituents R³, R⁴, R⁵, R⁶ and R⁷, independently of each other, are H, halogen, OH, Rₓ, ORₓ or COORₓ, wherein Rₓ is an optionally substituted and/or branched C₁ to C₁₀ alkyl residue,
X is CH₂, O, NH, NRₓ, S or SO₂,
n is the number 0, 1 or 2 and
wherein for substrates with formula (III) R¹ is not OH,
   which comprises the following steps:
a) providing at least one type of whole-cell catalyst, containing:
   i. a gene A which codes for the enzyme styrene monooxygenase and is under the functional control of a regulatable promoter,
   ii. a gene B which codes for the enzyme epoxide isomerase and is under the functional control of a regulatable promoter, and/or
   iii. a gene D which codes for the enzyme styrene oxide reductase, in conjunction with a gene E which codes for the enzyme alcohol dehydrogenase, wherein the genes D and E are under the functional control of a regulatable promoter,
in an aqueous component,
b) activating the whole-cell catalyst with an inducer and/or an activator, which results in the expression of the genes A, B and/or D and E,
c) bringing the activated whole-cell catalyst into contact with a substrate with formula (III) and/or (IV), wherein the substrate is reacted with at least one enzyme as defined in (a) to form a reaction product with formula (I) and/or (II),
d) isolating at least one reaction product with formula (I) and/or (II) which has been produced.

Surprisingly, it has been established that after the biocatalytic transformation of a substrate with general formula (III) or (IV) to form a reaction product with formula (I) or respectively (II), the reaction products are not metabolized further in the whole-cell catalysts (i.e. degraded by metabolization of the whole-cell catalyst) and accumulate in the aqueous component. Advantageously, the reaction products with formula (I) or respectively (II) accumulate in the aqueous component as a result of ejection from the whole-cell catalyst.

The basis of the present invention is the very recent recognition that substituted substrates with general formula (III) or (IV) are metabolized by styrene monooxygenase, epoxide isomerase, styrene oxide reductase and alcohol dehydrogenase.

Surprisingly, the enzyme phenylactyl-CoA ligase further down in the cell metabolism is only capable of using a substituted reaction product with formula (I) or respectively formula (II) to a limited extent or preferably to a zero extent for further metabolization and this reaction product accumulates in the aqueous component.

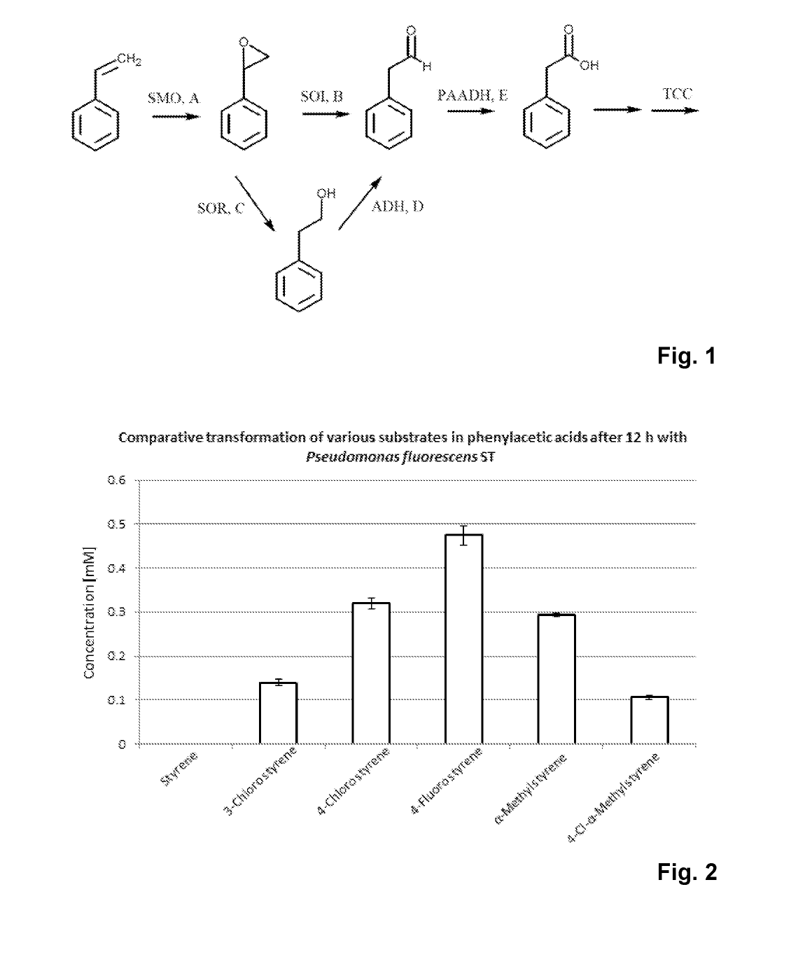

The method in accordance with the invention thus has the advantage that by using whole-cell catalysts, preferably two enzymes (styrene monooxygenase and epoxide isomerase, FIG. 1), particularly preferably all three enzymes (styrene monooxygenase, epoxide isomerase and aldehyde dehydrogenase, FIG. 1) can be used substantially for the biocatalytic synthesis of phenylacetaldehyde derivatives as a precursor of phenylacetic acid and/or its derivatives, particular preferably of phenylacetic acid and/or its derivatives in accordance with formula (I) or respectively (II), since the stability of the said three enzymes in the context of the biochemical synthesis is increased and contributes to more stable process management. By varying the process procedure as regards the form of substrate addition (preferably via the gas phase, directly to the medium, two-phase system), this can be adapted to the cell density and the organism used, whereupon an optimized biocatalytic transformation of substrates with general formula (III) or (IV) and a process run time which is as long as possible is permitted.

In a preferred embodiment of the invention, the whole-cell catalyst contains:
i. a gene A which codes for the enzyme styrene monooxygenase and is under the functional control of a regulatable promoter,
ii. a gene B which codes for the enzyme epoxide isomerase and is under the functional control of a regulatable promoter, and
iii. optionally, a gene C, which codes for the enzyme aldehyde dehydrogenase and is under the functional control of a regulatable promoter.

In an alternative preferred embodiment of the method of the invention, whole-cell catalysts are used in which, for the biocatalytic synthesis of phenylacetaldehyde derivatives as precursors of phenylacetic acid and/or its derivatives, particularly preferably of phenylacetic acid and/or its derivatives in accordance with formula (I) or respectively (II), the three enzymes styrene monooxygenase, styrene oxide reductase and alcohol dehydrogenase, particularly preferably the enzymes styrene monooxygenase, styrene oxide reductase, alcohol dehydrogenase and aldehyde dehydrogenase may be used substantially. Preferably, the whole-cell sensor contains:
i. a gene A which codes for the enzyme styrene monooxygenase and is under the functional control of a regulatable promoter,
ii. a gene D which codes for the enzyme styrene oxide reductase, in conjunction with a gene E which codes for the enzyme alcohol dehydrogenase, wherein the genes D and E are under the functional control of a regulatable promoter, and
iii. optionally, a gene C, which codes for the enzyme aldehyde dehydrogenase and is under the functional control of a regulatable promoter.

Styrene monooxygenase is an enzyme in bacteria which catalyses the chemical transformation of styrene with flavine adenine dinucleotide (FADH₂) in accordance with the reaction:

styrene+FADH₂+O₂ [symbol] (S)-2-phenyloxirane+FAD+H₂O

as the first step of the aerobic styrene degradation pathway (i.e. in the presence of oxygen) in bacteria to form the intermediate (S)-2-phenyloxiran (styrene oxide). FAD is regenerated by the reductase subunit of the styrene monooxygenase with the consumption of NADH.

Epoxide isomerase is a naturally occurring enzyme which belongs to the isomerase class and catalyses the chemical transformation of the intermediate styrene oxide into phenylacetaldehyde.

Styrene oxide reductase is an enzyme which transforms styrene oxide into 2-phenylethanol and in this respect is dependent on co-factors within the cell. Preferably, the co-factors are NADH or NADPH.

Alcohol dehydrogenase is an enzyme which catalyses the transformation of alcohols into aldehydes. The enzyme is dependent on co-factors. Preferably, the co-factors are NAD⁺, NADP⁺, and cytochrome. In addition, the enzyme is capable of implementing the reverse reaction as well.

Aldehyde dehydrogenase is an oxidoreductase and is selected from a group of enzymes which oxidize aldehydes to carboxylates in the metabolic functions of living organisms.

Epoxide isomerase is, for example, a co-factor (NADH and FAD)-independent enzyme, but in contrast, styrene monooxygenases are preferably co-factor-dependent, wherein FADH₂ is enzymatically transformed into FAD. However, in the context of biocatalytic reactions, the energy source FADH₂ is not lost, since it is regenerated via enzymatic transformation with an aldehyde dehydrogenase with the intermediate compound NADH.

Advantageously, the method of the invention concerns the synthesis of agents with general formula (I) and/or formula (II) using whole-cell catalysts which contain the genes A, B and C or respectively A, C, D, E or A, B, C, D, E, in order to form a so-called quasi co-factor-independent system, since the co-factors (NADH and FADH₂) required as energy sources are regenerated in situ.

The corresponding gene and amino acid sequences for the said enzymes (styrene monooxygenase, epoxide isomerase, alcohol dehydrogenases and aldehyde dehydrogenase) are very well known to the skilled person or can be obtained from known databases (for example NCBI; RCSB PDB; UniProt; PDB Europa).

The genes A, B, C, D and E, which code for the said enzymes, are under the functional control of a regulatable promoter, wherein the promoters may be identical to or different from each other.

In accordance with the invention, activation of the whole-cell catalyst is carried out in a signal-dependent manner by contacting with an activator and/or an inducer, wherein induction of expression of the genes A, B, C, D, E is signal-dependent and the whole-cell catalyst is transformed into its active form. Preferably, the activators or respectively inducers activate the regulatable promoter (in the context of the application also termed the operator), in that they interact directly with a regulatable promoter or in that they bind to a repressor protein which is then released from the promoter. By contacting the whole-cell catalyst with an activator and/or inducer, the enzymes mentioned above (styrene monooxygenase, epoxide isomerase, styrene oxide reductase, alcohol dehydrogenase and aldehyde dehydrogenase) are synthesized in the whole-cell catalyst and thus are available for the biocatalytic transformation of a substrate with formula (III) and/or (IV).

In one embodiment of the invention, the regulatable promoters are different from each other, so that the promoters can be activated in a primary signal-specific manner. Advantageously, in this manner the presence of different activators and/or inducers in the whole-cell catalyst can result in the expression of selected genes, whereupon the stress for a recombinant cell is minimized.

The corresponding nucleic acid sequences for promoters (for example the T7-Promotor in pET16 expression systems) which can be used for a method in accordance with the invention are very well known to the skilled person or may be obtained from known databases (for example EPD; TRED; MPromDB). Advantageously, in addition to promoters introduced into the organism, natural promoters may also be used for a method in accordance with the invention.

Preferably, the whole-cell catalysts are brought into contact with an activator and/or inducer in a concentration of the activator and/or inducer with respect to the total volume of the aqueous component in the range 1 to 1000 μM, particularly preferably in the range 10 to 800 μM, more particularly preferably in the range 25 to 500 μM, wherein the activator and/or inducer can be supplied continuously or discontinuously.

By bringing the whole-cell catalyst into contact with a substrate with formula (III) and/or (IV), a biocatalytic transformation to form a corresponding reaction product with formula (I) or respectively (II) takes place within a whole-cell catalyst with at least one enzyme from styrene monooxygenase, epoxide isomerase and/or aldehyde dehydrogenase. This means that substrates in accordance with the invention with formula (III) are biocatalytically transformed into reaction products with formula (I) or respectively substrates with formula (IV) are biocatalytically transformed into reaction products with formula (II).

Alternatively, by bringing the whole-cell catalyst into contact with a substrate with formula (III) and/or (IV) within a whole-cell catalyst having at least one of the enzymes styrene monooxygenase, styrene oxide reductase and alcohol dehydrogenase and/or aldehyde dehydrogenase, a biocatalytic transformation takes place to form a corresponding reaction product with formula (I) or respectively (II).

In accordance with the invention, the substrate with formula (III) and/or (IV) is resorbed by the whole-cell catalyst (i.e. taken up in the cell) and is biocatalytically transformed by means of at least one enzyme as described above selected from A, B, C, D and E to form a reaction product with formula (I) and/or (II).

Preferably, the whole-cell catalyst is brought into contact with a substrate with formula (III) and/or (IV) by introducing the substrate in the gas phase and/or by direct addition to the liquid components in the form of a liquid and/or solid. In addition, when directly added to the whole-cell biocatalyst, an organic phase may be used as a substrate reservoir, whereupon the processing time can be optimized.

In the case where the substrate is an agent with general formula (III) or respectively (IV), wherein R² is not H (i.e. R² is a linear or branched C₁ to C₃ alkyl residue), the corresponding reaction products with formula (I) or respectively (II) have a chiral centre (*) on the C atom with the residue R².

All enantiomers and racemic mixtures of reactions products with formula (I) and (II) can be formed by means of a method in accordance with the invention and thus may in principle be considered to be encompassed in the method of the invention.

In this regard, it has surprisingly been found that in the biocatalytic transformation of a substrate with general formula (III) or respectively (IV) wherein R² is not H, individual enantiomers, preferably in a defined configuration, more particularly preferably S- or R-configuration, are formed with a high enantiomeric excess. Preferably, the enantiomeric excess is at least 20%, particularly preferably at least 40%.

Alternatively, the reaction products with general formula (I) and/or (II) may be a racemic mixture with an enantiomeric excess in the range 0 to 20%, preferably in the range 0 to 10%, more particularly preferably in the range 0 to 5%.

The enantiomeric excess (ee in %) is defined as

$$ee ( \% ) = \frac{\vert ( R - S ) \vert}{R + S} \cdot 100$$

wherein R and S denote the molar concentration of the R- or S-configured enantiomer respectively and is always a positive value.

In accordance with the invention, when contacting activated whole-cell catalysts which contain the three genes A, B and C, substrates with formula (III) are used:
wherein R¹ is H or a linear C₁ to C₃ alkyl residue, particularly preferably H or methyl,
R² is H or a linear or branched C₁ to C₃ alkyl residue (methyl, ethyl, isopropyl, n-propyl),
R³, R⁴, R⁵, R⁶ and R⁷, independently of each other, are H, halogen, OH, Rₓ, ORₓ or COORₓ, particularly preferably H, halogen or Rₓ,
wherein Rₓ is an optionally substituted and/or branched C₁ to C₁₀ alkyl residue, particularly preferably a C₁ to C₅ alkyl residue, more particularly preferably methyl, ethyl, n-propyl, isopropyl or isobutyl,
   whereupon their corresponding acids or ketones (i.e. when R¹ is not H) in accordance with formula (I) are formed.

In accordance with the invention, when contacting activated whole-cell catalysts which contain the genes A, C, D and E, substrates with formula (III) are used:
wherein R¹ is H or a linear C₁ to C₃ alkyl residue, particularly preferably H or methyl,
R² is H or a linear or branched C₁ to C₃ alkyl residue (methyl, ethyl, isopropyl, n-propyl),
R³, R⁴, R⁵, R⁶ and R⁷, independently of each other, are H, halogen, OH, Rₓ, ORₓ or COORₓ, particularly preferably H, halogen, OH, ORₓ or Rₓ, more particularly preferably H, halogen or Rₓ,
wherein Rₓ is an optionally substituted and/or branched C₁ to C₈ alkyl residue, particularly preferably a C₁ to C₅ alkyl residue, more particularly preferably methyl, ethyl, n-propyl, isopropyl or isobutyl,
   wherein their corresponding acids or ketones (i.e. when R¹ is not H) in accordance with formula (I) are formed.

Substrates with formula (III) are particularly preferred, wherein two of the residues R³, R⁴, R⁵, R⁶ and R⁷ are a substituent other than H (halogen, OH, Rₓ, ORₓ or COORₓ); more particularly preferably, exclusively one of the residues R³, R⁴, R⁵, R⁶ and R⁷ is a substituent other than H.

In accordance with the invention, when contacting activated whole-cell catalysts which contain the two genes A and B substrates with formula (III) are used:
wherein R¹ is H or a linear C₁ to C₃ alkyl residue, particularly preferably H or methyl,
R² is H or a linear or branched C₁ to C₃ alkyl residue (methyl, ethyl, isopropyl or n-propyl),
R³, R⁴, R⁵, R⁶ and R⁷, independently of each other, are H, halogen, OH, Rₓ, ORₓ or COORₓ, particularly preferably H, halogen or Rₓ,
wherein Rₓ is an optionally substituted and/or branched C₁ to C₁₀ alkyl residue, particularly preferably a C₁ to C₅ alkyl residue, more particularly preferably methyl, ethyl, n-propyl, isopropyl and/or isobutyl,
   wherein their corresponding acids or ketones (i.e. R¹ is not H) in accordance with formula (I) are formed.

In accordance with the invention, when contacting activated whole-cell catalysts which contain the genes A, D and E, substrates with formula (III) are used:
wherein R¹ is H or a linear C₁ to C₃ alkyl residue, particularly preferably H or methyl,
R² is H or a linear or branched C₁ to C₃ alkyl residue (methyl, ethyl, isopropyl or n-propyl),
R³, R⁴, R⁵, R⁶ and R⁷, independently of each other, are H, halogen, OH, Rₓ, ORₓ or COORₓ, particularly preferably H, halogen, OH, ORₓ or Rₓ,
wherein Rₓ is an optionally substituted and/or branched C₁ to C₈ alkyl residue, particularly preferably a C₁ to C₅ alkyl residue, more particularly preferably methyl, ethyl, n-propyl, isopropyl and/or isobutyl,
   wherein their corresponding acids or ketones (i.e. R¹ is not H) in accordance with formula (I) are formed.

Particularly preferred substrates are those with formula (III), wherein two of the residues R³, R⁴, R⁵, R⁶ and R⁷ are a substituent other than H (halogen, OH, Rₓ, ORₓ or COORₓ); more particularly preferably, exclusively one of the residues R³, R⁴, R⁵, R⁶ and R⁷ is a substituent other than H.

In accordance with the invention, when contacting activated whole-cell catalysts which contain the genes A, B and/or C, preferably authentic bacterial cells, then bicyclic substrates with formula (IV) are used, wherein:
the substituent R² is a linear or branched C₁ to C₃ alkyl residue (methyl, ethyl, isopropyl or n-propyl),
the substituents R³, R⁴, R⁵ and R⁶, independently of each other, are H, halogen, OH, Rₓ, ORₓ or COORₓ, particularly preferably H, halogen, OH, ORₓ or Rₓ,
wherein Rₓ is an optionally substituted and/or branched C₁ to C₈ alkyl residue, particularly preferably a C₁ to C₅ alkyl residue, more particularly preferably methyl, ethyl, n-propyl, isopropyl and/or isobutyl,
X is a CH₂, O, NH, NRₓ, S or SO₂, particularly preferably CH₂, O, NH or NRₓ, more particularly preferably CH₂ or NH,
n is the number 0, 1 or 2, particularly preferably the number 0 or 1,
   wherein the corresponding bicyclic ketone with formula (II) are formed.

Particularly preferred substrates are those with formula (IV), wherein two of the residues R³, R⁴, R⁵ and R⁶ are a substituent other than H (halogen, OH, Rₓ, ORₓ or COORₓ); more particularly preferably, exclusively one of the residues R³, R⁴, R⁵ and R⁶ is a substituent other than H.

The substrate with formula (III) and/or (IV) is preferably used in a concentration in the range 0.1 to 10 mM, particularly preferably in the range 0.2 to 5 mM, more particularly preferably in the range 0.2 to 2.5 mM, in a biocatalytic transformation and may be used in a continuous or discontinuous manner.

Preferably, the quantity of reaction product with formula (I) or respectively (II) after the biocatalytic transformation of a substrate with formula (III) or respectively (IV) is at least 30% molar, particularly preferably at least 40% molar, more particularly preferably at least 50% molar of the quantity of the originally employed substrate.

It may be desirable for the substrate and the corresponding reaction product to be present in a defined ratio which differs from the ratio mentioned above. In this case, the reaction can be interrupted at any time.

Preferably, the reaction product with formula (I) or respectively (II) is secreted from the whole-cell catalyst into the aqueous component, whereupon isolation of the at least one reaction product with formula (I) or respectively (II) from the biomass and the aqueous component is promoted.

Preferably, the reaction product with formula (I) or respectively (II) is isolated from the biomass and the aqueous component in steps, wherein in a first step the biomass is separated from the aqueous component containing a reaction product with formula (I) or respectively (II) by centrifuging or filtration.

References given above and below are only provided insofar as they are necessary for the skilled person to understand the invention.

In a preferred embodiment of the method of the invention, one type of whole-cell catalyst is selected from recombinant (i.e. genetically modified) and/or authentic bacterial cells.

The methods for culturing recombinant and/or authentic bacterial cells are known to the skilled person, wherein the bacterial cells are continuously or discontinuously cultured in a batch method or a fed-batch method or repeated fed-batch method for the purposes of propagation or biocatalytic transformation of a substrate with general formula (III) or respectively formula (IV). A summary of known culture methods is given in the text book by Chmiel (Bioprozesstechnik 1. Einführung in die Bioverfahrenstechnik [Bioprocessing Technology 1. Introduction to Bioprocessing Technology] (Gustav Fischer Verlag, Stuttgart, 1991)) or in the text book by Storhas (Bioreaktoren and periphere Einrichtungen [Bioreactors and Peripheral Equipment] (Vieweg Verlag, Braunschweig/Wiesbaden, 1994)).

The aqueous component to be used must be suitable for the bacterial strains employed. Descriptions of aqueous components (for example culture media) for various microorganisms are described in the manual “Manual of Methods for General Bacteriology” from the American Society for Bacteriology (Washington D.C., USA, 1981).

The substances which can be used which are described in the mentioned publications (for example carbon sources, nitrogen sources, metallic salts) may be added to the aqueous components in the form of a one-off addition or as appropriate during culture. To control the pH of the aqueous component, basic compounds such as sodium hydroxide, potassium hydroxide, ammonia or ammoniacal solution, or acidic compounds such as phosphoric acid or sulphuric acid, for example, may be added in an appropriate manner and/or buffering agents such as hydrogen phosphate salts or TRIS may be used. In order to control foam formation, antifoaming agents such as fatty acid polyglycol esters, for example, may be used. In order to maintain the stability of the plasmids, appropriate substances with selective actions such as antibiotics (for example chloramphenicol, ampicillin, kanamycin) may be added to the aqueous component. Bacterial cells with partially inactivated metabolic pathways (for example auxotrophic mutations) are preferred, containing at least one gene A, B, D, E and/or C, including genes for completing incomplete metabolic pathways. In order to maintain aerobic conditions, oxygen or oxygen-containing gas mixtures such as air, for example, are introduced into the aqueous component.

Culturing (propagation) of the bacterial biomass in the form of bacteria can thus be obtained by the skilled person in known manner, for example by culturing in LB medium, but preferably, however, by culturing in a medium which enables the production of high cell densities, in particular more than 1×10⁹ cells per mL. Propagation is preferably carried out in the usual laboratory shaker flasks, but in order to produce larger quantities of bacterial biomass, propagation under controlled conditions in a fermenter is also possible.

Preferably, the authentic and/or recombinant bacterial cells are cultured under physiological conditions at a temperature in the range 0° C. to 60° C., preferably in the range 10° C. to 50° C., particularly preferably in the range 20° C. to 40° C., wherein the pH of the aqueous component is preferably in the range 5.8 to 8.5, particularly preferably in the range 6.8 to 8.0.

Preferably, authentic bacterial cells contain all three genes A, B and C which code for the enzymes styrene monooxygenase, epoxide isomerase and aldehyde dehydrogenase, wherein all three genes A, B and C are under the functional control of an identical promoter or operator. Alternatively, all three genes A, B and C are under the control of several promoters or operators.

Preferably, authentic bacterial cells from wild type strains are used for the method of the invention, wherein the wild type strains are selected from Rhodococcus, Pseudomonas, Sphingobium, Sphingopyxis and Corynebacterium, particularly preferably from Rhodococcus opacus 1CP, Rhodococcus species ST-5, Pseudomonas fluorescens ST, Corynebacterium species AC-5, Pseudomonas putida CA-3 and Pseudomonas putida S12. Particularly preferably again, the wild type strain is selected from Sphingopyxis sp. Kp5.2 (DSM 28731).

Alternatively and preferably, authentic bacterial cells contain the genes A, C, D and E, which code for the enzymes styrene monooxygenase, styrene oxide reductase and alcohol dehydrogenase, wherein all of the genes A, C, D and E are under the functional control of an identical promoter or operator. Alternatively and preferably, all of the genes A, C, D and E are under the functional control of several promoters or operators. In this regard, authentic bacterial cells from wild type strains are preferably used, wherein the genus Gordonia, particularly preferably Gordonia sp. CWB2 (DSM 46758) is particularly preferred.

Advantageously, the authentic bacterial cells used, which may be employed in a method in accordance with the invention for the biocatalytic synthesis of phenylacetic acid and/or its derivatives, are on the risk class 1 list of the ZKBS (Zentrale Kommission für die Biologische Sicherheit [Central Commission for Biological Safety]) and thus are designated as non-pathogenic for humans and animals. Since they constitute natural isolates, there is no requirement for them to be subject to gene technology licenses.

In a preferred embodiment of the method of the invention, the recombinant bacterial cells which can be used for the biocatalytic synthesis of agents with formula (I) and/or (II) from substrates with formula (III) and/or (IV) are negative mutations of authentic bacterial cells, or insertion mutations.

Preferably, the recombinant bacterial cells are negative mutations (i.e. knock-out-mutations or deletion mutations) of the wild type strains mentioned above (i.e. authentic bacterial cells which naturally comprise the three genes A, B and C), wherein a gene A, B and/or C, preferably the gene C. has been partially or completely deleted and/or has been exchanged for a modified gene. In the context of the invention, the term “negative mutation” is synonymous with the terms “deletion mutation” and “knock-out mutation”.

Alternatively and preferably, the recombinant bacterial cells are negative mutations (i.e. knock-out mutations or deletion mutations) of the wild type strains mentioned above (i.e. authentic bacterial cells which naturally comprise the genes A, C, D, E or A, B, C, D, E), wherein a gene A, B, D, E and/or C, preferably the gene C, has been partially or completely deleted and/or has been exchanged for a modified gene.

In the case in which unsubstituted phenylacetic acids are to be obtained, the recombinant bacterial cells are preferably negative mutations (i.e. knock-out mutations or deletion mutations) from which a gene which codes for phenylacetyl-CoA ligase has been partially or completely deleted.

Alternatively, recombinant bacterial cells are generated by introducing nucleotide sequences of the genes A, B, D, E and/or C into bacterial cells by genome insertion or the introduction of expression vectors, whereupon so-called insertion mutations are formed. Preferably, insertion mutations are not naturally suitable for the biocatalytic synthesis of agents with formula (I) and/or (II), since they originally did not contain a nucleotide sequence of the genes A, B, D, E and/or C. Potential host organisms for insertion mutations are preferably selected from the genuses Escherichia, Pseudomonas, Arthrobacter, Rhodococcus, Corynebacterium and Bacillus.

By deliberately inserting selected nucleotide sequences of the genes A, B, D, E and/or C into a bacterial cell, the reaction rates, the yields and the enantiomeric excess can be increased in the biocatalytic transformation of the invention of substrates with formula (III) and/or (IV). The nucleotide sequences of the genes A, B, D, E and C thus comprise authentic and/or artificial reading frames. Preferably, an artificial reading frame is adapted via gene synthesis to the “Codon Usage” of the host organism.

In a preferred embodiment of the invention, the nucleotide sequences to be inserted are designed such that they have nucleotide sequences between the genes A, B, D, E and/or C which code for authentic or artificial amino acid linkers so that recombinant enzymes in the form of heterodimers or heterotrimers are formed by expression, wherein the enzymes (styrene monooxygenase, epoxide isomerase, styrene oxide reductase, alcohol dehydrogenase and/or aldehyde dehydrogenase) are covalently bound together via linker sequences.

In an alternative preferred embodiment of the invention, the nucleotide sequences to be inserted are designed such that they have nucleotide sequences between the genes A, B, D, E and/or C which code for authentic or artificial amino acid linkers so that recombinant enzymes in the form of heterodimers, heterotrimers, heterotetramers or heteropentamers are formed by expression, wherein the enzymes (styrene monooxygenase, epoxide isomerase, styrene oxide reductase, alcohol dehydrogenase and/or aldehyde dehydrogenase) are covalently bound together via linker sequences.

In principle, suitable genes A, B, D, E and/or C are amplified using known methods such as the polymerase chain reaction (PCR) with the aid of short synthetic nucleotide sequences (primers) and then isolated. In general, the primers used are produced with the aid of known gene sequences based on existing homologies with the genes A, B, D, E and/or C.

Ideally, the vector for cloning an amplified gene A, B, D, E and/or C has a small molecular mass and contains selectable genes for resulting in an easily recognized phenotype in a cell so that a simple selection of vector-containing and vector-free host cells is possible. In order to obtain a high yield of DNA and corresponding gene products, the vector should comprise a strong promoter and/or regulatory sequence. In addition, an origin of replication is important for replication of the vector. As an example, pET vector systems based on an antibiotic selection are suitable.

When using authentic bacterial cells as the whole-cell catalyst, the inducer and/or activator is preferably selected from styrene, styrene oxide and/or phenylacetaldehyde, more particularly preferably from styrene and/or styrene oxide.

Preferably, the epoxide isomerase is a styrene oxide-isomerase with EC No: 5.3.99.7 and the aldehyde dehydrogenase is a phenylacetaldehyde dehydrogenase with EC No: 1.2.1.39. Advantageously, the alcohol dehydrogenase is a 2-phenylethanol dehydrogenase with EC No 1.1.1.

Preferably, the reaction product with formula (I) or respectively (II) is isolated by extraction of the aqueous component with an organic solvent selected from the group formed by phthalic acid esters, particularly preferably bis(2-ethylhexyl)phthalate, 1,2-cyclohexanedicarbonic acid diisononylester and Mesamoll®, and/or aliphatic linear and/or branched hydrocarbons, preferably containing 5 to 16 carbon atoms, such as n-pentane, cyclopentane, n-hexane, cyclohexane, n-heptane, n-octane, cyclooctane, n-decane, n-dodecane or n-hexadecane, for example. Preferably, said organic solvents are used for extraction in a single-phase aqueous system after transformation of a substrate with formula (III) or respectively (IV). Alternatively, said organic solvents are used in a two-phase system in the form of a second phase in addition to the aqueous component as a reservoir for a substrate with formula (III) or respectively (IV) and/or for separation of reaction products with formula (I) and (II), preferably of substituted or unsubstituted ketones and/or bicyclic derivatives, more particularly preferably of bicyclic derivatives with formula (II).

Moreover, halogenated aliphatic hydrocarbons are suitable for extraction of the product after transformation, preferably containing one or two carbon atoms such as, for example, dichloromethane, chloroform, carbon tetrachloride, dichloroethane or tetrachloroethane, aliphatic acyclic and cyclic ethers, preferably containing 4 to 8 carbon atoms such as, for example, diethylether, methyl-tert-butylether, ethyl-tert-butylether, dipropylether, diisopropylether, dibutylether, tetrahydrofuran or esters such as, for example, ethylacetate or n-butylacetate or ketones such as, for example, methylisobutylketone or dioxane, or mixtures thereof.

The reaction product with formula (I) or respectively (II) is advantageously isolated by extraction of the aqueous component with an organic solvent preferably after separation of the whole-cell catalyst in the form of biomass from the aqueous component, wherein separation of the whole-cell catalyst in the form of biomass is preferably carried out from the aqueous component by means of centrifuging or filtration.

Preferably, extraction of the reaction products with formula (I), wherein R₁═OH, and with formula (II) is carried out with an organic solvent at a pH in the range 0 to 8, particularly preferably in the range 1 to 7, more particularly preferably in the range 2 to 6, wherein advantageously, high extraction ratios can be obtained. By definition, the extraction ratio is a measure of the efficiency of the extraction and provides information as to how much product (in g) has been taken up by the organic solvent with respect to the total quantity of product. The higher this value, the better an organic solvent extracts the product. In a preferred embodiment, the extraction ratio is greater than 4:1, particularly preferably greater than 6:1 and most preferably greater than 8:1.

If appropriate, purification of the reaction product with formula (I) and/or (II) is carried out subsequent to extraction and is carried out by distillation, wherein preferably, the organic solvent is separated out. Preferably, separation of the organic solvent is carried out by evaporation at a pressure in the range 0.1 to 1000 mbar, particularly preferably in the range 0.1 to 750 mbar, more particularly preferably in the range 1 to 400 mbar.

Alternatively to the extraction of products with formula (I) wherein R₁═OH from the aqueous component with an organic solvent, extraction of the aqueous component may also be carried out with pH-dependent methods for solid phase extraction. As an example, after producing an alkaline pH in the liquid component, anion exchangers or, for acidic pHs, hydrophobic adsorbent resins may be used as the adsorber.

Preferably, the biocatalytic synthesis of substituted or unsubstituted phenylacetic acids and/or substituted or unsubstituted ketones and/or their bicyclic derivatives with formula (I) and/or (II) is carried out in a single-phase aqueous system or in a two-phase system.

In a preferred embodiment of the invention, the biocatalytic method for the synthesis of phenylacetic acids and/or its derivatives with formula (I) and/or (II) is carried out in a two-phase system. In this regard, organic solvents as mentioned above or ionic liquids, both of which are substantially immiscible with water, are used as the second organic phase, wherein preferably, the substrate accumulates in the organic phase. Examples of known two-phase systems are described in the publications by Panke et al. (Biotechnol. Bioeng. 2000, 69, 91-100) and Wubbolts et al. (Enzyme Microb. Technol. 1994, 16, 887-894).

The term “substantially water-immiscible organic phases” means organic phases which contain less than 1% by weight, preferably less than 0.5% by weight of water with respect to the total weight of the organic phases.

Preferably, agents with formula (III) are used as substrates for the biocatalytic synthesis of substituted or unsubstituted phenylacetic acids and/or ketones with formula (I), wherein:
the substituent R¹ is H or a linear C₁ to C₃ alkyl residue,
the substituent R² is H or a linear or branched C₁ to C₃ alkyl residue,
the substituents R³, R⁴, R⁵, R⁶ and R⁷ independently of each other, are H, halogen, OH or Rₓ, wherein Rₓ is an optionally substituted and/or branched C₁ to C₅ alkyl residue, more particularly preferably methyl, ethyl, n-propyl, isopropyl or isobutyl,
   wherein exclusively a maximum of two of the residues R³, R⁴, R⁵, R⁶ and R⁷ are a substituent other than H (halogen, OH, Rₓ, ORₓ or COORₓ); more particularly preferably, exclusively one of the residues R³, R⁴, R⁵, R⁶ and R⁷ is a substituent other than H.

More particularly preferred substrates with general formula (III) are:
2-fluorostyrene, 3-fluorostyrene or 4-fluorostyrene, 2-fluoro-α-alkylstyrene, 3-fluoro-α-alkylstyrene, 4-fluoro-α-alkylstyrene
2-chlorostyrene, 3-chlorostyrene or 4-chlorostyrene, 2-chloro-α-alkylstyrene, 3-chloro-α-alkylstyrene, 4-chloro-α-alkylstyrene
2-bromostyrene, 3-bromostyrene or 4-bromostyrene, 2-bromo-α-alkylstyrene, 3-bromo-α-alkylstyrene, 4-bromo-α-alkylstyrene
2-iodostyrene, 3-iodostyrene or 4-iodostyrene, 2-iodo-α-alkylstyrene, 3-iodo-α-alkylstyrene, 4-iodo-α-alkylstyrene
2-isobutyl-α-alkylstyrene, 3-isobutyl-α-alkylstyrene, 4-isobutyl-α-alkylstyrene
2-methylstyrene, 3-methylstyrene or 4-methylstyrene, 2-methyl-α-alkylstyrene, 3-methyl-α-alkylstyrene, 4-methyl-α-alkylstyrene
2-methoxy-4-vinylphenol
3,4-methylenedioxy styrene
   wherein the term “alkyl” denotes a linear or branched C₁ to C₃ alkyl residue.

Preferably, agents with formula (IV) are used as bicyclic substrates for the biocatalytic synthesis of substituted or unsubstituted bicyclic derivatives with general formula (II), wherein:
the substituent R² is H or a linear or branched C₁ to C₃ alkyl residue (methyl, ethyl, isopropyl or n-propyl),
the substituents R³, R⁴, R⁵ and R⁶, independently of each other, are H, halogen, OH or Rₓ, wherein Rₓ may be a substituted and/or branched C₁ to C₅ alkyl residue, more particularly preferably methyl, ethyl, n-propyl, isopropyl or isobutyl,
X is a CH₂, O, NH or NRₓ, more particularly preferably CH₂ or NH,
n is the number 0, 1 or 2, particularly preferably the number 0 or 1.
   wherein exclusively a maximum of two of the residues R³, R⁴, R⁵ and R⁶ are a substituent other than H (halogen, OH, Rₓ, ORₓ or COORₓ); more particularly preferably, exclusively one of the residues R³, R⁴, R⁵ and R⁶ is a substituent other than H.

As an example, a substrate with general formula (IV) is more particularly preferably:
an indole (which reacts further to form indigo as the final product, cf. O'Connor et al. [Appl. Environ. Microbiol. 1997, 63, 4287-4291])
an indene.

Particularly advantageously, the following representatives of reaction products with general formula (I) and (II) may be biocatalytically synthesized using the method of the invention:
4-chloro-, 4-fluoro- and 4-methyl-phenylacetic acid (in particular capable of being produced with Pseudomonas fluorescens ST and Sphingopyxis sp. Kp.5.2)
4-hydroxy-3-methoxy phenylacetic acid (homovanillic acid) and 4-hydroxy-3-methoxy phenylacetaldehyde (in particular producible with Pseudomonas fluorescens ST and Gordonia sp. CWB2)
α-methylphenylacetic acid and 4-chloro-α-methylphenylacetic acid (in particular producible with Pseudomonas fluorescens ST and Sphingopyxis sp. Kp.5.2)
(RS)-2-(4-isobutylphenyl)propionic acid (in particular producible with Gordonia sp. CWB2)
derivatives of 3,4-methylenedioxyphenylacetaldehyde and 3,4-methylenedioxy-phenylacetic acid.

It should be noted that the embodiments of the invention may be combined in any order.

In a particularly preferred embodiment of the method of the invention for the synthesis of substituted phenylacetic acids with general formula (I), wherein:
R¹ is OH,
R² is H,
the substituents R³, R⁴, R⁵, R⁶ and R⁷, independently of each other, are H, OH or ORₓ, wherein R, is an optionally substituted and/or branched C₁ to 05 alkyl residue, more particularly preferably methyl, ethyl, n-propyl, isopropyl or isobutyl,
   in particular for the synthesis of derivatives of 4-hydroxyphenylacetic acid, (for example 4-hydroxy-3-methoxy phenylacetic acid (also known as homovanillic acid), derivatives of 3-hydroxyphenylacetic acid or derivatives of 2-hydroxyphenylacetic acid,
   these are prepared from appropriately substituted substrates with general formula (III) obtained by:
a) providing a whole-cell catalyst, containing:
   i. a gene A which codes for the enzyme styrene monooxygenase and is under the functional control of a regulatable promoter,
   ii. a gene B which codes for the enzyme epoxide isomerase and is under the functional control of a regulatable promoter, and
   iii. a gene C, which codes for the enzyme aldehyde dehydrogenase and is under the functional control of a regulatable promoter.
      in an aqueous component,
b) activating the whole-cell catalyst with an inducer and/or an activator, which results in the expression of the genes A, B and C,
c) bringing the whole-cell catalyst into contact with the substrate with formula (III), wherein the substrate is transformed with at least one enzyme as defined in (a) to form the reaction product with formula (I).

Alternatively and preferably, the whole-cell catalyst of the method of the invention for the synthesis of substituted phenylacetic acids with general formula (I), wherein:
R¹ is OH,
R² is H,
the substituents R³, R⁴, R⁵, R⁶ and R⁷, independently of each other, are H, OH or ORₓ, wherein Rₓ is an optionally substituted and/or branched C₁ to C₅ alkyl residue, more particularly preferably methyl, ethyl, n-propyl, isopropyl or isobutyl,
   in particular for the synthesis of derivatives of 4-hydroxyphenylacetic acid, (for example 4-hydroxy-3-methoxy phenylacetic acid (also known as homovanillic acid), derivatives of 3-hydroxyphenylacetic acid or derivatives of 2-hydroxyphenylacetic acid, contains
i. a gene A which codes for the enzyme styrene monooxygenase and is under the functional control of a regulatable promoter,
ii. a gene D which codes for the enzyme styrene oxide reductase, in conjunction with a gene E which codes for the enzyme alcohol dehydrogenase, wherein the genes D and E are under the functional control of a regulatable promoter, and
iii. optionally, a gene C, which codes for the enzyme aldehyde dehydrogenase and is under the functional control of a regulatable promoter.

Preferably, the reaction product with formula (I) in the whole-cell catalysts are not further limited or preferably not further metabolized (i.e. degraded by metabolization of the whole-cell catalyst) and accumulate in the aqueous components. Advantageously, the reaction product with formula (I) accumulates as a result of ejection from the whole-cell catalyst into the aqueous component, whereupon preferably at least one of the reaction products with formula (I) which is formed is isolated.

Preferably, whole-cell catalysts in an aqueous component are prepared in the method of the invention with an OD₆₀₀ in the range 0.5 to 30.

Preferably, the whole-cell catalyst is activated by contact with an activator and/or an inducer by direct addition to the aqueous component in the form of a liquid and/or solid or via the gas phase, whereupon the whole-cell catalyst is transformed into its active form. Preferably, contacting with the activator and/or inducer is carried out for a reaction period in the range 1 to 96 hours, particularly preferably 12 to 72 hours, wherein the activator and/or the inducer can be added continuously or discontinuously.

Preferably, contacting of the whole-cell catalyst with the substrate with formula (III) and/or (IV) is carried out after activation of the whole-cell catalyst via the gas phase and/or by direct addition to the liquid component in the form of a liquid and/or solid, whereupon the substrate is resorbed (i.e. taken up) by the whole-cell catalyst and biocatalytically transformed with at least one enzyme as described above selected from A, B, C, D and E, to form a reaction product with formula (I) and/or (II). Advantageously, contacting of the whole-cell catalyst with a substrate with formula (III) and/or (IV) is carried out in portions.

The invention also encompasses recombinant bacterial cells, preferably negative mutations and/or insertion mutations for the biocatalytic synthesis of substituted or unsubstituted phenylacetic acid and/or its cyclic derivatives in accordance with formula (III) and/or formula (IV) containing:
i. a gene A which codes for the enzyme styrene monooxygenase and is under the functional control of a regulatable promoter,
ii. a gene B which codes for the enzyme epoxide isomerase and is under the functional control of a regulatable promoter, and
iii. optionally, a gene C, which codes for the enzyme aldehyde dehydrogenase and is under the functional control of a regulatable promoter.

The invention also encompasses recombinant bacterial cells, preferably negative mutations and/or insertion mutations for the biocatalytic synthesis of substituted or unsubstituted phenylacetic acid and/or its cyclic derivatives in accordance with formula (III) and/or formula (IV) containing:
i. a gene A which codes for the enzyme styrene monooxygenase and is under the functional control of a regulatable promoter,
ii. the gene D, which codes for the enzyme styrene oxide reductase, in conjunction with a gene E which codes for the enzyme alcohol dehydrogenase, under the functional control of a regulatable promoter,
iii. optionally, a gene C, which codes for the enzyme aldehyde dehydrogenase and is under the functional control of a regulatable promoter.

Preferably, the recombinant bacterial cells are negative mutations of authentic bacterial cells or insertion mutations.

In a preferred embodiment of the invention, the genes A, B and/or C of the recombinant bacterial cells contain artificial reading frames. More preferably, the genes D and/or E of the recombinant bacterial cells contain artificial reading frames.

Preferably, the regulatable promoters of the recombinant bacterial cells are different, so that the promoters can be primary signal-specifically activated. Advantageously, therefore, the presence of different activators and/or inducers in the whole-cell catalyst can result in the expression of selected genes A, B, D, E and/or C, whereupon stress as a result of gene expression is minimized for a recombinant cell. Commercial systems based on the lac-operon, on T7-promoters, on trp- and phoA- as well as on araB regulators are also suitable, inter alia, wherein induction can be carried out depending on the system with IPTG, tryptophan, by phosphate depletion or with arabinose.

The invention also pertains to a kit for the biocatalytic synthesis of substituted or unsubstituted phenylacetic acids and/or ketones and/or their bicyclic derivatives in accordance with formula (I) and/or formula (II) containing:
a) at least one type of whole-cell catalysts, preferably one type of recombinant bacterial cells in an aqueous component and/or
b) at least one type of cryopreserved whole-cell catalysts, preferably one type of recombinant bacterial cells.

The bacterial biomass in the form of bacteria for a method in accordance with the invention for biocatalytic synthesis may be obtained in a manner which is known to the skilled person for preculture of the whole-cell catalyst contained in a kit in accordance with the invention (propagation on full medium and minimum medium), for example by culture in full medium, such as LB medium (DSM-Medium No. 381), advantageously however by culture in a medium which enables the production of high cell densities, for example by culture in minimum medium such as DSM-Medium No. 55), in particular of more than 1×10⁹ cells per mL. Propagation of these whole-cell catalysts containing preculture of a kit in accordance with the invention is preferably carried out in the usual laboratory shaker flasks in order to produce larger quantities of bacterial biomass; in addition, propagation under controlled conditions in a fermenter is possible.

After propagating bacterial biomass in the form of bacteria for a method for biocatalytic synthesis in accordance with the invention by preculture of the whole-cell catalysts contained in a kit in accordance with the invention (propagation on full medium and minimum medium), this biomass may be transferred into the aqueous component for biocatalytic synthesis of substituted or unsubstituted phenylacetic acids and/or ketones and/or their bicyclic derivatives in accordance with formula (I) and/or formula (II).

The invention also relates to authentic bacterial cells for the biocatalytic synthesis of substituted or unsubstituted phenylacetic acids and/or ketones and/or their bicyclic derivatives in accordance with formula (I) and/or formula (II), wherein the authentic bacterial cells are selected from Rhodococcus, Pseudomonas, Sphingobium, Sphingopyxis and Corynebacterium, particularly preferably from Rhodococcus opacus 1CP, Rhodococcus species ST-5, Pseudomonas fluorescens ST, Corynebacterium species AC-5, Pseudomonas putida CA-3 and Pseudomonas putida S12. The wild type strain from Sphingopyxis sp. Kp5.2 (DSM 28731) is also particularly preferred. The strain Kp5.2 (DSM 28731) was deposited on 30.04.2014 at the Deutschen Stammsammlung von Mikroorganismen and Zellkulturen GmbH [German Microorganism and Cell Culture Strain Collection] (DSMZ, Mascheroder Weg Ib, D-38124 Braunschweig), under the auspices of the “Budapester Vertrag über die internationale Anerkennung der Hinterlegung von Mikroorganismen für die Zwecke von Patentverfahren” [Budapest Treaty on the International Recognition of the Deposit of Microorganisms for the Purpose of Patent Procedure].

The strain Kp5.2 (DSM 28731) is characterized by:
i. a gene A which codes for the enzyme styrene monooxygenase and is under the functional control of a regulatable promoter,
ii. a gene B which codes for the enzyme epoxide isomerase and is under the functional control of a regulatable promoter, and
iii. a gene C, which codes for the enzyme aldehyde dehydrogenase and is under the functional control of a regulatable promoter.

Alternatives are the authentic bacterial cells Gordonia, particularly preferably Gordonia sp. CWB2 (DSM 46758). The strain Gordonia sp. CWB2 (DSM 46758) was deposited on 30.04.2014 at the Deutschen Stammsammlung von Mikroorganismen and Zellkulturen GmbH [German Microorganism and Cell Culture Strain Collection] (DSMZ, Mascheroder Weg Ib, D-38124 Braunschweig), under the auspices of the “Budapester Vertrag über die internationale Anerkennung der Hinterlegung von Mikroorganismen für die Zwecke von Patentverfahren” [Budapest Treaty on the International Recognition of the Deposit of Microorganisms for the Purpose of Patent Procedure].

The strain CWB2 (DSM 46758) is characterized by:
i. a gene A which codes for the enzyme styrene monooxygenase and is under the functional control of a regulatable promoter,
ii. the gene D, which codes for the enzyme styrene oxide reductase, in conjunction with a gene E which codes for the enzyme alcohol dehydrogenase, and is under the functional control of a regulatable promoter.

It has been established that by providing the novel bacterial strain Gordonia sp. CWB2 (DSM 46758) in a method in accordance with the invention for the synthesis of substituted or unsubstituted phenylacetic acids and/or ketones and/or their bicyclic derivatives in accordance with formula (I) and/or formula (II), advantageously substituted or unsubstituted phenylacetic acids and/or ketones, wherein Rₓ is an optionally substituted and/or branched C₁ to C₈ alkyl residue, particularly preferably a C₁ to C₅ alkyl residue, more particularly preferably methyl, ethyl, n-propyl, isopropyl and/or isobutyl, can be synthesized.

The invention also relates to the use of recombinant bacterial cells, preferably negative mutations and/or insertion mutations, for a method in accordance with the invention or a kit in accordance with the invention.

The invention also pertains to an aqueous component containing at least one reaction product with formula (I) or respectively (II), obtained by the method in accordance with the invention as described above, comprising:
a) providing at least one type of whole-cell catalyst, containing:
   i. a gene A which codes for the enzyme styrene monooxygenase and is under the functional control of a regulatable promoter,
   ii. a gene B which codes for the enzyme epoxide isomerase and is under the functional control of a regulatable promoter, and/or
   iii. a gene D which codes for the enzyme styrene oxide reductase, in conjunction with a gene E which codes for the enzyme alcohol dehydrogenase, and are under the functional control of a regulatable promoter,
      in an aqueous component,
b) activating the whole-cell catalyst with an inducer and/or an activator, which results in the expression of the genes A, B and/or D and E,
c) contacting the whole-cell catalyst with a substrate with formula (III) and/or (IV), wherein the substrate is reacted with at least one enzyme as defined in (a) to form a reaction product with formula (I) and/or (II),
   wherein the reaction products in the whole-cell catalysts are not metabolized further (i.e. degraded during metabolization of the whole-cell catalyst) and accumulate in the aqueous component.

The following figures and exemplary embodiments are intended to explain the invention in more detail without in any way limiting its scope.

FIG. 1: Metabolization of styrene by side chain oxidation with the enzymes styrene monooxygenase (SMO), styrene oxide isomerase (SOI) and phenylacetaldehyde dehydrogenase (PAADH), or monooxygenase (SMO), styrene oxide reductase (SOR), alcohol dehydrogenase (ADH) and phenylacetaldehyde dehydrogenase (PAADH), wherein the phenylacetic acid formed is supplied via subsequent intermediate steps of the tricarboxylic acid cycle (TCA cycle) (based on Velasco et al. [J. Bacteriol. 1998, 180, 1063-1071], modified; O'Leary et al. [FEMS Microbiol. Rev. 2002, 26, 403-417], modified).

FIG. 2: Transformation of substituted styrenes using Pseudomonas fluorescens ST as the authentic whole-cell catalyst, wherein the product concentrations [mM] are shown after 12 hours starting from 1.25 mM of substrate.

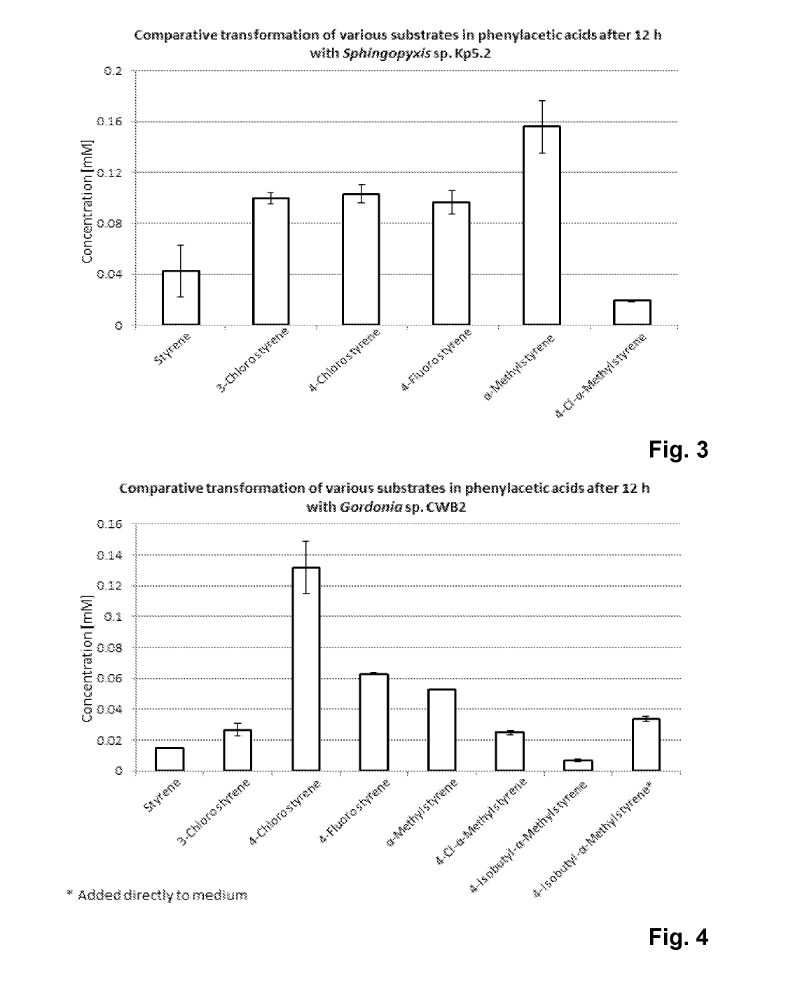

FIG. 3: Transformation of substituted styrenes using Sphingopyxis sp. Kp5.2 as the authentic whole-cell catalyst, wherein the product concentrations [mM] are shown after 12 hours starting from 1.25 mM of substrate.

FIG. 4: Transformation of substituted styrenes using Gordonia sp. CWB2 (DSM 46758) as the authentic whole-cell catalyst, wherein the product concentrations [mM] are shown after 12 hours starting from 1.25 mM of substrate.

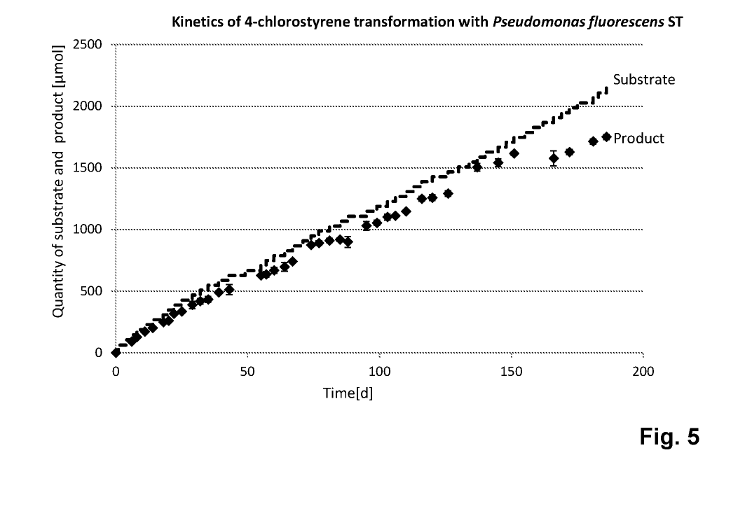

FIG. 5: Transformation of 4-chlorostyrene using Pseudomonas fluorescens ST as the authentic whole-cell catalyst, wherein the quantities [μmol] of substrate and product are shown over a time period of 186 days.

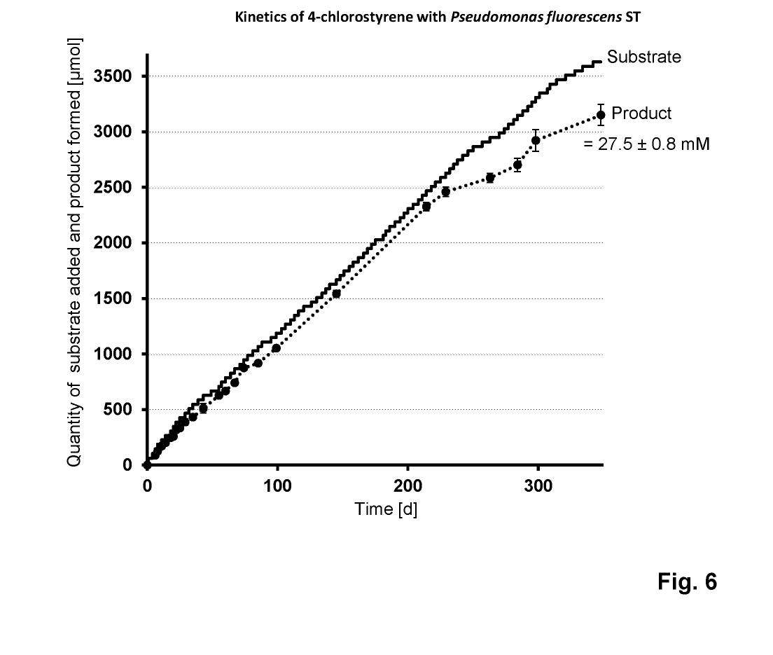

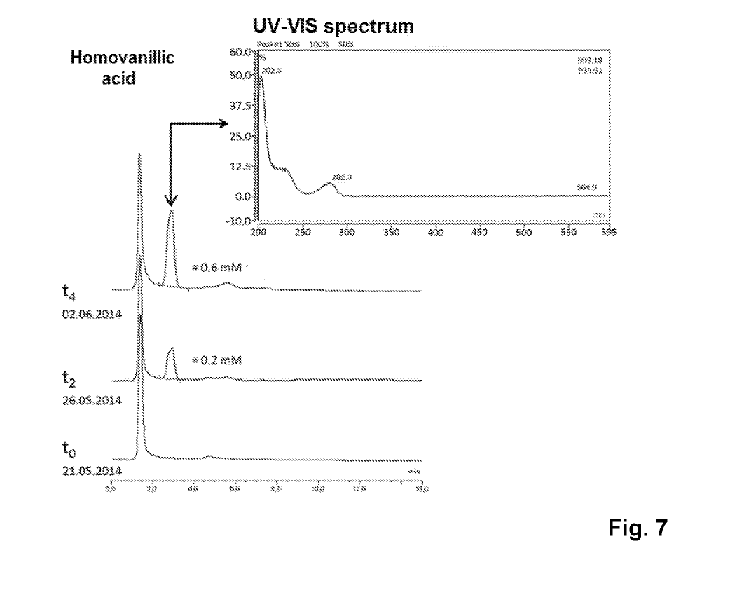

FIG. 6: Reaction of 4-chlorostyrene using Pseudomonas fluorescens ST as the authentic whole-cell catalyst, wherein the quantities [μmol] of substrate and product are shown over a time period of 348 days.

FIG. 7: HPLC chromatograms and UV-VIS product spectrum for the reaction of 4-vinylguaiacol in homovanillic acid using Pseudomonas fluorescens ST as the authentic whole-cell catalyst.

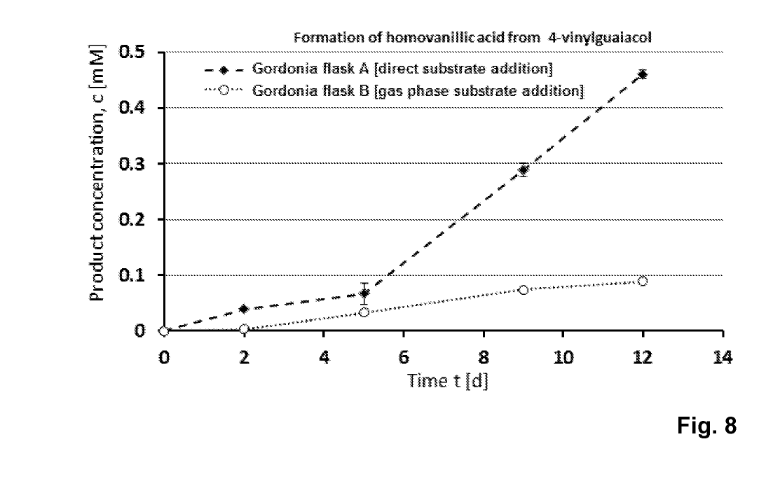

FIG. 8: Transformation of 4-vinylguaiacol in homovanillic acid using Gordonia sp. CWB2 as the authentic whole-cell catalyst, wherein the product concentrations [mM] obtained are shown over 12 days.

Unless otherwise indicated, in the following implementational examples, the culture of whole-cell catalysts was carried out on minimum medium (modified in accordance with Dorn, E.; Hellwig, M.; Reineke, W.; Knackmuss, H.-J. (1974) Isolation and characterization of a 3-chlorobenzoate-degrading pseudomonad. Arch Microbiol 99: 61-70), which was composed of the following strain solutions autoclaved separately from each other:

| | | |
|---|---|---|
| 20 × phosphate buffer | 100 | mL |
| Na₂HPO₄•2H₂O | 70 | g |
| KH₂PO₄ | 20 | g |
| H₂O (deionized) | ad 1 | l |
| 50 × salt solution | 20 | mL |
| Ca(NO₃)₂•4H₂O | 3 | g |
| Fe(III)NH₄-citrate | 0.5 | g |
| MgSO₄•7H₂O | 10 | g |
| (NH₄)₂SO₄ | 50 | g |
| 1000 × trace element solution 6 | 50 | mL |
| (Pfennig & Lippert, 1966) | | |
| H₂O (deionized) | ad 1 | l |
| Carbon source (strain solution | x | mL |
| or pure component) | | |
| H₂O (deionized) | ad 1 | l |

Glucose or fructose were used as the carbon source. In addition, in order to improve the growth in the liquid medium, yeast extract was sometimes added in a final concentration of 0.07% to 0.1% (w/v).

### EXAMPLE 1

### Synthesis of Substituted Phenylacetic Acids with Pseudomonas fluorescens ST

1 L flasks with 200 mL of minimum medium (Dorn et al. 1974) and an initial amount of 0.05% yeast as well as 5 mM of glucose (as the carbon source) were inoculated with a preculture of a type of whole-cell catalyst (Pseudomonas fluorescens ST) and then the biomass was cultured with glucose to an OD₆₀₀ (optical density at a wavelength of 600 nm) of approximately 1.5. Next, the biomass was induced for at least 3 days with daily additions of 17-26 pmol of styrene (as inducer); beforehand, each of the flasks was aerated. The styrene was added in the gas phase using an evaporator unit. Next, the cells were harvested by centrifuging at 4° C. and 5000×g (30 min). The pellet was then washed twice with 50 mL of a 25 mM phosphate buffer solution (pH=7) and then taken up in a suitable quantity of phosphate buffer (25 mM; pH=7). Next, the various substrates were added by means of an evaporator unit via the gas phase. The transformation was preferably carried out at 30° C. and 120 rpm.

In a preliminary experiment with a cell suspension (OD₆₀₀=1; dry mass of cells approximately 0.6 mg/mL) with a single addition of 1.25 mM of substrate respectively (supplied via the gas chamber unless otherwise stated) after incomplete transformation of the substrate within 12 h, the product concentrations in the culture medium shown in Table 1 and FIG. 2 were detected. It should be noted that this example concerned a test transformation in order to examine the spectrum of substrates which could be transformed by one type of whole-cell catalyst. The yields given are thus not the final yields as obtained after completion of a method in accordance with the invention (see implementational examples 4 and 5).

TABLE 1
12-hour yields for transformation of styrenes with
cells of Pseudomonas fluorescens ST

| | | Product | Incomplete |
|---|---|---|---|
| | | concen- | yield [%] |
| | | tration | after |
| Substrate | Product | [μM] | 12 h |
| styrene | phenylacetic acid | 0 | 0 |
| 3-chlorostyrene | 3-chlorophenylacetic acid | 140 | 11.2 |
| 4-chlorostyrene | 4-chlorophenylacetic acid | 320 | 25.6 |
| 4-fluorostyrene | 4-fluorophenylacetic acid | 475 | 38.0 |
| α-methylstyrene | α-methylphenylacetic | 290 | 23.2 |
| | acid | | |
| 4-chloro-α- | 4-chloro-α-methylphenyl- | 105 | 8.4 |
| methylstyrene | acetic acid | | |

### EXAMPLE 2

### Synthesis of Substituted Phenylacetic Acids with the Isolate Sphingopyxis sp. Kp5.2

1 L flasks with 200 mL of minimum medium (Dorn et al. 1974) and an initial amount of 0.05% yeast as well as 5 mM of glucose (as the carbon source) were inoculated with a preculture of a type of whole-cell catalyst (Sphingopyxis sp. Kp5.2) and then the biomass was cultured with glucose to an OD₆₀₀ of approximately 0.8. Next, the biomass was induced for at least 3 days with daily additions of 18-26 pmol of styrene (as inducer); beforehand, each of the flasks was aerated. The styrene was added in the gas phase using an evaporator unit. Next, the cells were harvested by centrifuging at 4° C. and 5000×g (30 min). The pellet was then washed twice with 50 mL of a 25 mM phosphate buffer solution (pH=7) and then taken up in a suitable quantity of phosphate buffer (25 mM; pH=7). Next, the various substrates were added by means of an evaporator unit via the gas phase. The transformation was preferably carried out at 30° C. and 120 rpm.

In a preliminary experiment with a cell suspension (OD₆₀₀=1; dry mass of cells approximately 1.0 mg/mL) with a single addition of 1.25 mM of substrate respectively (supplied via the gas chamber unless otherwise stated) after incomplete transformation of the substrate within 12 h, the product concentrations in the culture medium shown in Table 2 and FIG. 3 were detected. It should be noted that this example concerned a test transformation in order to examine the spectrum of substrates which could be transformed by one type of whole-cell catalyst. The yields given are thus not the final yields as obtained after completion of a method in accordance with the invention (see implementational examples 4 and 5).

TABLE 2
12-hour yields for transformation of styrenes with
cells of Sphingopyxis sp. Kp5.2

| | | Product | Incomplete |
|---|---|---|---|
| | | concen- | yield [%] |
| | | tration | after |
| Substrate | Product | [μM] | 12 h |
| styrene | phenylacetic acid | 43 | 3.4 |
| 3-chlorostyrene | 3-chlorophenylacetic acid | 100 | 8.0 |
| 4-chlorostyrene | 4-chlorophenylacetic acid | 102 | 8.2 |
| 4-fluorostyrene | 4-fluorophenylacetic acid | 97 | 7.8 |
| α-methylstyrene | α-methylphenylacetic | 156 | 12.5 |
| | acid | | |
| 4-chloro-α- | 4-chloro-α-methylphenyl- | 19 | 1.5 |
| methylstyrene | acetic acid | | |

### EXAMPLE 3

### Synthesis of Substituted Phenylacetic Acids with Gordonia sp. CWB2

1 L flasks with 200 mL of minimum medium (Dorn et al. 1974) and an initial amount of 0.05% yeast as well as 5 mM of glucose (as the carbon source) were inoculated with a preculture of a type of whole-cell catalyst (Gordonia sp. CWB2) and then the biomass was cultured with glucose to an OD₆₀₀ (optical density at a wavelength of 600 nm) of approximately 4.5. Next, the biomass was induced for at least 3 days with daily additions of 18-26 pmol of styrene (as inducer); beforehand, each of the flasks was aerated. The styrene was added in the gas phase using an evaporator unit. Next, the cells were harvested by centrifuging at 4° C. and 5000×g (30 min). The pellet was then washed twice with 50 mL of a 25 mM phosphate buffer solution (pH=7) and then taken up in a suitable quantity of phosphate buffer (25 mM; pH=7). Next, the various substrates were added via the gas phase. The transformation was preferably carried out at 30° C. and 120 rpm.

In a preliminary experiment with a cell suspension (OD₆₀₀=6.08; dry mass of cells approximately 1.5 mg/mL) with a single addition of 1.25 mM of substrate respectively (supplied via the gas chamber unless otherwise stated) after incomplete transformation of the substrate within 12 h, the product concentrations in the culture medium shown in Table 3 and FIG. 4 were detected. It should be noted that this example concerned a test transformation in order to examine the spectrum of substrates which could be transformed by one type of whole-cell catalyst. The yields given are thus not the final yields as obtained after completion of a method in accordance with the invention (see implementational examples 4 and 5).

TABLE 3
12-hour yields for transformation of styrenes
with cells of Gordonia sp. CWB2

| | | Product | Incomplete |
|---|---|---|---|
| | | concen- | yield [%] |
| | | tration | after |
| Substrate | Product | [μM] | 12 h |
| styrene | phenylacetic acid | 15 | 1.2 |
| 3-chlorostyrene | 3-chlorophenylacetic acid | 27 | 2.2 |
| 4-chlorostyrene | 4-chlorophenylacetic acid | 132 | 10.6 |
| 4-fluorostyrene | 4-fluorophenylacetic acid | 63 | 5.0 |
| α-methylstyrene | α-methylphenylacetic | 53 | 4.2 |
| | acid | | |
| 4-chloro-α- | 4-chloro-α-methylphenyl- | 25 | 2.0 |
| methylstyrene | acetic acid | | |
| 4-isobutyl-α- | 4-isobutyl-α-methylphenyl- | 7 | 0.6 |
| methylstyrene | acetic acid | | |
| | (Ibuprofen) | | |
| 4-isobutyl-α- | 4-isobutyl-α-methylphenyl- | 34 | 2.7 |
| methylstyrene | acetic acid | | |
| (direct addition to | (Ibuprofen) | | |
| medium) | | | |

### EXAMPLE 4

### Long Duration Experiment for the Synthesis of 4-Chloro-Phenylacetic Acid with Pseudomonas fluorescens ST

A 1 L flask with 200 mL of minimum medium (Dorn et al. 1974) and an initial amount of 0.05% yeast as well as 5 mM of glucose was inoculated with a preculture of a type of whole-cell catalyst (Pseudomonas fluorescens ST) and then the biomass was cultured with the addition of glucose to an OD₆₀₀ of 1. Next, the content of the flask was sterilely harvested by centrifuging (4° C., 5000×g, 30 min), the pellet was washed with sterile water or 25 mM phosphate buffer (pH=7) and then re-suspended in a suitable volume of minimum medium. Next, the aqueous component was incubated for 6 days with the whole-cell catalysts in the presence of styrene (as inducer). In this example, induction with styrene was carried out only after the harvesting and the washing steps, but may also have been carried out before that. Styrene was supplied via the gas phase using an evaporator unit (approximately 17-26 μmol every 1-3 days; the flask was aerated before each fresh supply). Next, over a period of several months, in addition to approximately 17 μmol of styrene (as the energy source and inducer), the substrate 4-chlorostyrene was added in portions of 20-40 μmol via the gas phase. The 4-chlorophenylacetic acid reaction product could be detected in the aqueous component.

In a preliminary experiment with a cell suspension (OD₆₀₀=0.8; dry mass of cells approximately 0.4-0.5 mg/mL), after 20 days a quantity of 260 μmol of reaction product could be obtained after adding 308 μmol of substrate, corresponding to a yield of 85%. After 60 days, the quantity of phenylacetic acid formed was approximately 670 μmol after adding 750 μmol of substrate (yield >85%). In 120 days, 1260 μmol of reaction product has been transformed from 1390 μmol of substrate, corresponding to a yield of up to 90%. The concentration obtained was 8.4 mM. The transformation profile is shown in FIG. 5. The gradual divergence between the supplied substrate and the product formed is due to an inactivation of the whole-cell catalyst, but over the total observation period it was not very pronounced.

### EXAMPLE 5

### Long Duration Experiment for the Synthesis of 4-Chloro-Phenylacetic Acid with Pseudomonas fluorescens ST (Longer Experimental Period)

A 1 L flask with 200 mL of minimum medium, an initial amount of 0.05% yeast as well as 5 mM of glucose was inoculated with a preculture of a type of whole-cell catalyst (Pseudomonas fluorescens ST) and then the biomass was cultured with the addition of glucose to an OD₆₀₀ of 1. Next, the content of the flask was sterilely harvested by centrifuging (4° C., 5000×g, 30 min), the pellet was washed with sterile water or 25 mM phosphate buffer (pH=7) and then re-suspended in a suitable volume of minimum medium. Next, the aqueous component was incubated for 6 days with the whole-cell catalysts in the presence of styrene (as inducer). In this example, induction with styrene was carried out only after the harvesting and the washing step, but may also have been carried out before that. Styrene was supplied via the gas phase using an evaporator unit (approximately 18-26 μmol every 1-3 days; the flask was aerated before each fresh supply). Next, over a period of several months, in addition to approximately 19-20 μmol of styrene (as the energy source and inducer), the substrate 4-chlorostyrene was added in portions of 21-42 μmol via the gas phase. The 4-chlorophenylacetic acid reaction product could be detected in the aqueous component.

In a preliminary experiment with a cell suspension (OD₆₀₀=0.8; dry mass of cells approximately 0.4 mg/mL), after 214 days a quantity of 2330 μmol of reaction product could be obtained after adding 2430 μmol of substrate, corresponding to a yield of 96%. After 284 days, the quantity of phenylacetic acid formed was approximately 2700 μmol after adding 3110 μmol of substrate (yield >87%). In 348 days, 3150 μmol of reaction product had finally been transformed from 3630 μmol of substrate, corresponding to a yield of approximately 87%. The concentration obtained was 27.5 mM. The transformation profile is shown in FIG. 6. The gradual divergence between the supplied substrate and the product formed is due to an inactivation of the whole-cell catalyst, but over the total observation period it was not very pronounced.

### EXAMPLE 6

### Stereoselective Reaction of 4-Chloro-α-Methylstyrene with Pseudomonas fluorescens ST

A whole-cell catalyst type was cultured and obtained as described in Examples 4 and 5. After induction of the biomass with styrene via the gas phase, in addition to approximately 17 μmol of styrene (as energy source and inducer), the substrate 4-chloro-α-methylstyrene was then added via the gas phase in portions of 20-40 μmol over a period of several days. The reaction product, 4-chloro-α-methylphenylacetic acid, could be detected in the culture medium.

In a preliminary experiment with a cell suspension (OD₆₀₀=0.8; dry mass of cells approximately 0.4-0.5 mg/mL), after 14 days a quantity of approximately 80 μmol of reaction product could be detected after adding 220 μmol of substrate, corresponding to a yield of approximately 36%. The concentration obtained in this regard was 0.4 mM. The reaction had an enantiomeric excess (ee) of 40% and was therefore enantiomer-selective. The literature regarding the enzymes involved did not predict this excess for the whole-cell catalyst used here.

### EXAMPLE 7

### Transformation of 4-Vinylguaiacol in Homovanillic Acid with Pseudomonas fluorescens ST

A 500 mL flask with 100 mL of minimum medium and 12.5 mM of glucose was inoculated with a preculture of a type of whole-cell catalyst (Pseudomonas fluorescens ST) and biomass was cultured by adding glucose to an OD₆₀₀ of 3.7. Next, the aqueous component was incubated with the whole-cell catalysts for a further day in the presence of styrene (as inducer). Styrene was supplied via the gas phase using an evaporator unit (approximately 13 μmol). Next, over a period of several days, in addition to approximately 13 μmol of styrene (as the energy source and inducer; fed in every 2-3 days; before each fresh supply, the flasks were aerated), the substrate 4-vinylguaiacol was added directly to the medium in portions of 50 μmol (150 μmol in total). The homovanillic acid reaction product could be detected in the aqueous component.

In a preliminary experiment with the cell suspension used (OD₆₀₀=3.7; dry mass of cells approximately 2.0 mg/mL), after 12 days a yield of approximately 40% homovanillic acid (0.6 mM, 11 mg in the pellet) was obtained (FIG. 7). The reaction was also highly selective. In addition to the target product, there was only one other, weak by-product which had a retention time of 5.5 min (see FIG. 7).

### EXAMPLE 8

### Transformation of 4-Vinylguaiacol in Homovanillic Acid with Gordonia sp CWB2

A 1 L flask each with 200 mL of minimum medium and 5 mM of fructose was inoculated with a preculture of a type of whole-cell catalyst (Gordonia sp. CWB2 (DSM 46758)) and biomass was cultured by adding fructose to an OD₆₀₀ of approximately 5.9. Next, each aqueous component was incubated with the whole-cell catalysts for a further two days in the presence of styrene (as inducer). Styrene was supplied via the gas phase using an evaporator unit (twice with approximately 26 μmol, on the second day the flask was aerated before the fresh supply). Next, over a period of several days, in addition to approximately 26 μmol of styrene (as the energy source and inducer; added every 2-3 days; before each fresh supply, the flasks were aerated), the substrate 4-vinylguaiacol was added in portions of 100 μmol once directly into the medium (flask A), once supplied via the gas phase (flask B) (300 μmol substrate in total). The homovanillic acid reaction product could be detected in each aqueous component.

In a preliminary experiment with the cell suspension used (OD₆₀₀ approximately 5.9; dry mass of cells approximately 1.9 mg/mL), after 12 days a yield of approximately 33% homovanillic acid (0.5 mM, 18 mg in the pellet) was obtained in flask A (FIG. 8). The reaction was also highly selective. 0.1 mM of homovanillic acid was formed in flask B, corresponding to a yield of 7% (FIG. 8). Thus, in the case of 4-vinylguaiacol, then, direct addition of the substrate to the medium is significantly preferred.

1. A method for the biocatalytic synthesis of substituted or unsubstituted compounds in accordance with formula (I) and/or their bicyclic derivatives in accordance with formula (II), 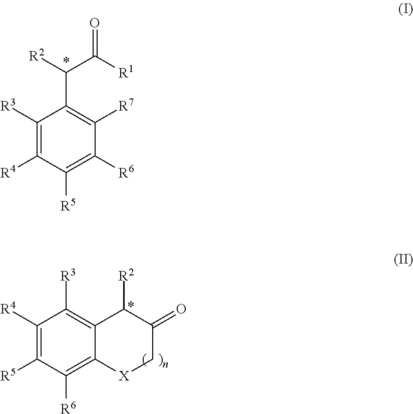
by means of the biocatalytic transformation of a substrate with formula (III) and/or formula (IV): 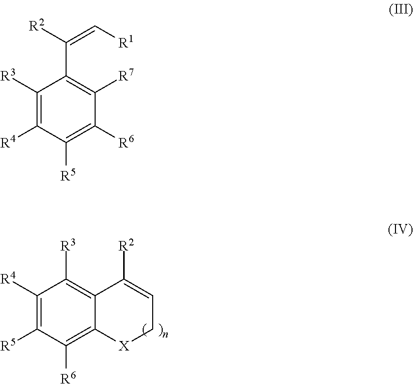
wherein:
the substituent R¹ is H, OH or a linear or branched C₁ to C₃ alkyl residue,
the substituent R² is H or a linear or branched C₁ to C₃ alkyl residue, wherein * is a chiral centre,
the substituents R³, R⁴, R⁵, R⁶ and R⁷ independently of each other, are H, halogen, OH, Rₓ, ORₓ or COORₓ, wherein Rₓ is an optionally substituted and/or branched C₁ to C₁₀ alkyl residue,
X is CH₂, O, NH, NRₓ, S or SO₂,
n is the number 0, 1 or 2,
the method comprising:
a) providing at least one whole-cell catalyst, comprising:
i. a gene A which codes for the enzyme styrene monooxygenase and is under the functional control of a regulatable promoter;
ii. a gene B which codes for the enzyme epoxide isomerase and is under the functional control of a regulatable promoter; and/or
iii. a gene D which codes for the enzyme styrene oxide reductase, in conjunction with a gene E which codes for the enzyme alcohol dehydrogenase, wherein the genes D and E are under the functional control of a regulatable promoter,
in an aqueous component;
b) activating the whole-cell catalyst with an inducer and/or an activator, which results in the expression of the genes defined in (a);
c) contacting the whole-cell catalyst with a substrate with formula (III) and/or (IV), wherein the substrate is reacted with at least one enzyme as defined in (a) to form a reaction product with formula (I) and/or (II); and
d) isolating at least one reaction product with formula (I) and/or (II) which has been produced.

2. The method according to claim 1, wherein the whole-cell catalyst comprises:
i. a gene A which codes for the enzyme styrene monooxygenase and is under the functional control of a regulatable promoter, and
ii. a gene B which codes for the enzyme epoxide isomerase and is under the functional control of a regulatable promoter; or
i. a gene A which codes for the enzyme styrene monooxygenase and is under the functional control of a regulatable promoter, and
ii. a gene D which codes for the enzyme styrene oxide reductase, in conjunction with a gene E which codes for the enzyme alcohol dehydrogenase, wherein the genes D and E are under the functional control of a regulatable promoter.

3. The method according to claim 2, the whole-cell catalyst further comprising:
a gene C, which codes for the enzyme aldehyde dehydrogenase and is under the functional control of a regulatable promoter.

4. The method according to claim 1, wherein the whole-cell catalyst is selected from authentic bacterial cells, recombinant bacterial cells, or combination thereof.

5. The method according to claim 1 wherein the whole-cell catalyst is authentic bacterial cells selected from Rhodococcus, Pseudomonas, Sphingobium, Sphingopyxis, and Corynebacteriium.

6. The method according to claim 1, wherein the whole-cell catalyst is authentic bacterial cells selected from Gordonia.

7. The method according to claim 4, wherein the recombinant bacterial cells are negative mutations of authentic bacterial cells or insertion mutations.

8. The method according to claim 1, wherein the inducer is one or more of styrene, styrene oxide, or phenylacetaldehyde.

9. The method according to claim 1, wherein the epoxide isomerase is a styrene oxide-isomerase and the aldehyde dehydrogenase is a phenylacetaldehyde dehydrogenase.

10. The method according to claim 1, wherein the product is isolated by extraction with an organic solvent or by means of solid phase extraction.

11. The method according to claim 1, wherein the biocatalytic synthesis of agents with formula (I) and/or formula (II) is carried out in a single-phase aqueous system or in a two-phase system.

12. The method according to claim 1, wherein the agents with formula (III) are used as the substrate, wherein:
the substituent R¹ is H or a linear or branched C₁ to C₃ alkyl residue,
the substituent R² is H or a linear or branched C₁ to C₃ alkyl residue,
the substituents R³, R⁴, R⁵, R⁶ and R⁷, independently of each other, are H, halogen, OH or Rₓ, wherein Rₓ is a C₁ to C₅ alkyl residue,
wherein a maximum of two of the residues R³, R⁴, R⁵, R⁶ and R⁷ are a substituent other than H.

13. The method according to claim 1, wherein the agents with formula (IV) are used as the bicyclic substrate, wherein:
the substituent R² is H or a linear or branched C₁ to C₃ alkyl residue;
the substituents R³, R⁴, R⁵ and R⁶, independently of each other, are H, halogen, OH or Rₓ, wherein Rₓ is a C₁ to C₅ alkyl residue;
X is a CH₂, O, NH or NRₓ; and
n is the number 0, 1 or 2,
wherein a maximum of two of the residues R³, R⁴, R⁵ and R⁶ are a substituent other than H.

14. The method according to claim 1, wherein the enantiomeric excess of the reaction product is at least 70%.

15. Recombinant bacterial cells for the biocatalytic synthesis of substituted or unsubstituted phenylacetic acids and/or ketones and/or their bicyclic derivatives in accordance with formula (I) and/or formula (II) 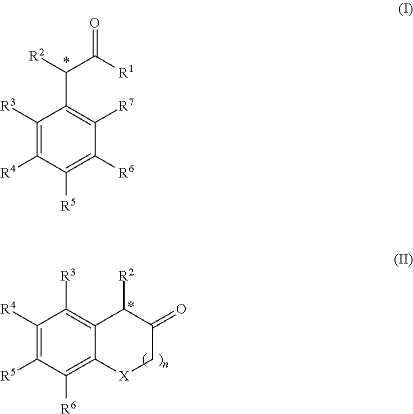
the recombinant bacterial cells comprising:
i. a gene A which codes for the enzyme styrene monooxygenase and is under the functional control of a regulatable promoter, and
ii. a gene B which codes for the enzyme epoxide isomerase and is under the functional control of a regulatable promoter; or
i. a gene A which codes for the enzyme styrene monooxygenase and is under the functional control of a regulatable promoter, and
ii. a gene D which codes for the enzyme styrene oxide reductase, in conjunction with a gene E which codes for the enzyme alcohol dehydrogenase, wherein the genes D and E are under the functional control of a regulatable promoter.

16. The recombinant bacterial cells according to claim 15 further comprising:
a gene C, which codes for the enzyme aldehyde dehydrogenase and is under the functional control of a regulatable promoter.

17. The recombinant bacterial cells according to claim 15, wherein the recombinant bacterial cells are negative mutations of authentic bacterial cells or insertion mutations.

18. The recombinant bacterial cells according to claim 15, wherein the regulatable promoters differ from each other so that the promoters are primary signal-specifically activatable.

19. A kit for the biocatalytic synthesis of substituted or unsubstituted phenylacetic acids and/or ketones and/or their bicyclic derivatives in accordance with formula (I) and/or formula (II) 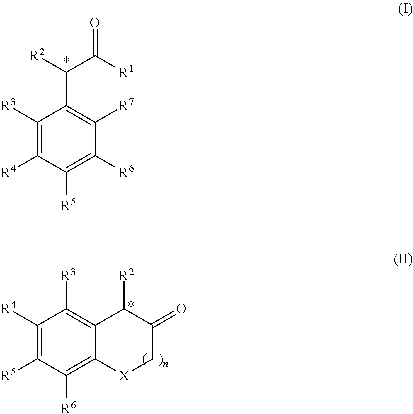
the kit comprising:
a) at least one type of recombinant bacterial cells according to claim 15 in an aqueous component; and/or
b) at least one type of cryopreserved, recombinant bacterial cells according to claim 15.

20.-22. (canceled)

23. The bacterial strain Sphingopyxis sp. Kp5.2 (DSM 28731).

24. The bacterial strain Gordonia sp. CWB2 (DSM 46758).

25. The recombinant bacterial cells according to claim 16, wherein the recombinant bacterial cells are negative mutations of authentic bacterial cells or insertion mutations.

26. The recombinant bacterial cells according to claim 16, wherein the regulatable promoters differ from each other so that the promoters are primary signal-specifically activatable.
